(19) United States
(12) Patent Application Publication
(10) Pub. No.: US 2015/0005382 A1
(43) Pub. Date: Jan. 1, 2015

(54) Methods for Identifying and Treating an Individual with an Inflammatory Disease using Fatty Acid-Based Therapies

(71) Applicant: Wake Forest University Health Sciences (WFUHS), Winston-Salem, NC (US)

(72) Inventor: Floyd H. Chilton, Clemmons, NC (US)

(21) Appl. No.: 14/367,158

(22) PCT Filed: Oct. 4, 2012
(86) PCT No.: PCT/US2012/058629

§ 371 Date: Jun. 19, 2014

Related U.S. Application Data
(60) Provisional application No. 61/542,973, filed on Oct. 4, 2011.

(51) Int. Cl. C12Q 1/68; A61K 36/30; A61K 31/202

(52) U.S. Cl. CPC C12Q 1/6883; A61K 31/202; A61K 36/30; C12Q 2600/156; C12Q 2600/106
USPC 514/560; 435/6.11

(57) ABSTRACT

The present invention provides methods of identifying a subject as responsive to treatment of an inflammatory disease and/or inflammation with one or more polyunsaturated fatty acids (PUFAs), comprising detecting, in a nucleic acid sample of a subject having an inflammatory disease and/or inflammation, the presence of a C allele at single nucleotide polymorphism (SNP) rs730012 in the leukotriene C4 synthase gene, wherein the presence of said C allele at rs730012 in said nucleic acid sample from the subject indicates that the subject is responsive to treatment of said inflammatory disease and/or inflammation with one or more PUFAs.

### STATEMENT OF PRIORITY

This application claims the benefit, under 35 U.S.C. §119 (e), of U.S. Provisional Application No. 61/542,973, filed Oct. 4, 2011, the entire contents of which are incorporated by reference herein.

### STATEMENT OF GOVERNMENT SUPPORT

The present invention was made with government support under grant number P50 AT0027820 awarded by the National Institutes of Health. The United States Government has certain rights in this invention.

### FIELD OF THE INVENTION

The present invention relates to a single nucleotide polymorphism in the leukotriene C4 synthase gene (A-444C) and the use of the leukotriene C4 synthase gene polymorphism for identifying whether a subject having an inflammatory disease and/or inflammation will be responsive or not to treatment with polyunsaturated fatty acids.

### BACKGROUND OF THE INVENTION

Research over the past 50 years has shown that both systemic and localized inflammation play an important role in the onset and progression of numerous chronic human diseases. There is also evidence that the incidence of people with chronic, low-grade and localized inflammation has markedly increased over the last 4 decades. This increase is thought to be a result of several factors including a dramatic escalation in the incidence of overweight/obesity and marked dietary changes as a result of shifts in the types of foods consumed in developed counties such as the United States.

These disturbing trends have created a new generation of patients who are seeking ways to maintain wellness and prevent the onset of these diseases, or to manage them after onset. According to the 2007 National Interview Survey Annual Report, 38% of adult Americans are using complementary and alternative medicine modalities. The most commonly used modalities are natural products enriched with polyunsaturated fatty acids (PUFAs), in particular ω-3 PUFAs, accounting for the largest proportion of usage (37% of natural products). Omega-3 PUFAs listed in this survey include both botanical based (flax seed and echium oils) and marine-based oils (fish oils). In addition to ω-3 PUFAs, there are also a number of ω-6 PUFA-based oils (e.g., borage and safflower oil) that have been shown to be beneficial for treating inflammatory disorders.

In spite of the overwhelming evidence for the beneficial effects of ω-3 PUFAs, the consumption of fish oil or other sources of ω-3 PUFAs in the North American population is still relatively low. For example, it is estimated that actual dietary intakes of fatty acid in fish oil are as low as one-tenth of the levels recommended by the American Heart Association. Over the past decade, research has shown that that there is great variability in the efficacy with which different individuals respond to both fish oil-based and/or botanical-based supplements, which may be one reason that the use of these supplements has not been widespread. Furthermore, to date it has been impossible to know in advance which patients will or will not respond to a PUFA-based therapy.

Accordingly, the present invention addresses the deficiencies in the art by providing methods for identifying subjects who will be responsive to treatment of an inflammatory disease with polyunsaturated fatty acid-based treatments (including dietary supplements, medical foods and prescription products).

### SUMMARY OF THE INVENTION

Quite unexpectedly, the present inventor has determined that genotyping a single SNP (-444) on the LTC4S (leukotriene C4 synthase) locus at rs730012 on chromosome 5 (5q35) for the C (A-444C) variant can predict the efficacy of polyunsaturated fatty acid-based treatments for inflammatory diseases and/or inflammation.

Thus, in one aspect, the present invention provides a method of identifying a subject as responsive to treatment of an inflammatory disease with one or more polyunsaturated fatty acids (PUFAs), comprising detecting, in a nucleic acid sample of a subject having an inflammatory disease, the presence of a C allele at single nucleotide polymorphism (SNP) rs730012 in the leukotriene C4 synthase gene, wherein the presence of said C allele at rs730012 in said nucleic acid sample from the subject indicates that the subject is responsive to treatment of said inflammatory disease with one or more PUFAs.

In another aspect, the present invention provides a method of identifying, within a population of subjects having an inflammatory disease, a subpopulation of subjects having an inflammatory disease that is responsive to treatment with one or more polyunsaturated fatty acids (PUFAs), comprising detecting, in a nucleic acid sample from each of said subjects, the presence of a C allele at single nucleotide polymorphism (SNP) rs730012, wherein the subpopulation of subjects having an inflammatory disease that is responsive to treatment with one or more PUFAs comprises those subjects having a C allele at rs730012, thereby identifying, within a population of subjects having an inflammatory disease, a subpopulation of subjects having an inflammatory disease that is responsive to treatment with one or more PUFAs.

Also provided herein is a method of determining a suitable treatment for an inflammatory disease in a subject in need thereof, comprising detecting, in a nucleic acid sample of the subject, the presence of a C allele at single nucleotide polymorphism (SNP) rs730012 in the leukotriene C4 synthase gene, wherein the C allele at rs730012 is correlated with responsiveness to treatment with one or more polyunsaturated fatty acids (PUFAs), thereby identifying a subject for which treatment with one or more PUFAs is a suitable treatment.

In a further aspect, the present invention provides a method of treating an inflammatory disease in a subject in need thereof with one or more polyunsaturated fatty acids (PUFAs), comprising (a) detecting, in a nucleic acid sample from the subject, the presence of a C allele at single nucleotide polymorphism (SNP) rs730012 in the leukotriene C4 synthase gene, wherein the presence of said C allele at rs730012 in said nucleic acid sample identifies the subject as being responsive to treatment of said inflammatory disease with one or more PUFAs; and (b) administering an effective amount of one or more PUFAs to the subject identified as responsive to treatment with one or more PUFAs, thereby treating an inflammatory disease in a subject in need thereof.

In an additional aspect, the present invention provides a method of treating inflammation in a subject in need thereof with one or more polyunsaturated fatty acids (PUFAs), comprising (a) detecting, in a nucleic acid sample from the subject, the presence of a C allele at single nucleotide polymorphism (SNP) rs730012 in the leukotriene C4 synthase gene, wherein the presence of said C allele at rs730012 in said nucleic acid sample identifies the subject as being responsive to treatment of said inflammation with one or more PUFAs; and (b) administering an effective amount of one or more PUFAs to the subject identified as responsive to treatment with one or more PUFAs, thereby treating inflammation in a subject in need thereof.

In another aspect, the present invention provides a method of preventing an inflammatory disease or inflammation in a subject, comprising (a) detecting, in a nucleic acid sample from the subject, the presence of a C allele at single nucleotide polymorphism (SNP) rs730012 in the leukotriene C4 synthase gene, wherein the presence of said C allele at rs730012 in said nucleic acid sample identifies the subject as being responsive to treatment of said inflammatory disease or inflammation with one or more PUFAs; and (b) administering an effective amount of one or more PUFAs to the subject identified as responsive to treatment with one or more PUFAs, thereby treating said inflammatory disease or inflammation in said subject.

In some embodiments, a polyunsaturated fatty acid is a botanical-based ω-3 PUFA, a marine-based ω-3 PUFA, a botanical-based ω-6 PUFA, or any combination thereof. In other embodiments of the invention, a botanical-based ω-3 PUFA is stearidonic acid (SDA), alpha-linolenic acid (ALA), eicosapentaenoic acid (EPA), docosapentaenoic acid (DPA), or any combination thereof, a marine based ω-3 PUFA is eicosapentaenoic acid (EPA), docosapentaenoic acid (DPA), docosahexaenoic acid (DHA), or any combination thereof, and a botanical-based ω-6 PUFA is gamma-linolenic acid (GLA), dihomogamma-linolenic acid (DGLA), or any combination thereof.

Other and further objects, features and advantages would be apparent and more readily understood by reading the following specification and by reference to the accompanying drawing forming a part thereof, or any examples of the embodiments of the invention given for the purpose of the disclosure.

### BRIEF DESCRIPTION OF THE FIGURES

FIG. 1 shows lung responsiveness to botanical oils in asthmatic individuals having the A allele of rs730012 versus those having the C allele of rs730012. Pearson chi2(1)=7.3684; P value=0.007.

FIG. 2 shows the level of expression of mRNA for the leukotriene C4 synthase gene in asthmatic individuals having the A allele of rs730012 versus those having the C allele of rs730012.

FIG. 3 shows the nucleotide sequence of the leukotriene C4 synthase gene (GenBank Accession No. U50136; SEQ ID NO:1). The first codon (ATG) is underlined (nucleotides 1447-1149) and the position of the A→C transversion 444 nucleotides upstream of the first codon is indicated by an underlined capital A allele

FIG. 4 provides a 20 nucleotide fragment of the leukotriene C4 synthase gene (SEQ ID NO:2) in which the position of the A→C transversion is indicated at nucleotide 13 by an underlined capital A.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the present invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications and patent applications mentioned in this specification are indicative of the level of skill of those skilled in the art that this invention pertains. Further, publications, patent applications, patents, and other references mentioned herein are incorporated by reference in their entirety.

The present invention is based on the unexpected discovery of a correlation between the presence of a single nucleotide polymorphism in the promoter region of the leukotriene C4 synthase gene (LTC4S) (i.e., an adenine to cytosine transversion 444 nucleotides upstream from the first codon in the leukotriene C4 synthase gene at rs730012) in a subject having an inflammatory disease and/or inflammation and responsiveness of the subject's inflammatory disease and/or inflammation to treatment with one or more polyunsaturated fatty acids (PUFAs). Thus, in one aspect, the present invention provides a method for predicting the responsiveness of a subject having an inflammatory disease to treatment with one or more PUFAs (e.g., marine and/or botanical based PUFAs) by detecting the presence of an A>C single nucleotide polymorphism (SNP) in the promoter region of the leukotriene C4 synthase gene (LTC4S), the SNP being located 444 nucleotides upstream from the first codon in the leukotriene C4 synthase gene at rs730012.

The nucleotide sequence of the leukotriene C4 synthase gene is provided as GenBank Accession No. U50136 (also provided herein as SEQ ID NO:1) and is 4441 nucleotides in length. The adenine to cytosine transversion polymorphism is at a position that is 444 nucleotides upstream from the first codon (ATG) (nucleotides 1447-1149) of the leukotriene C4 synthase gene. Thus, in addition to being described as a SNP at rs 730012, the SNP in the leukotriene C4 synthase gene can also be described as an A→C transversion at the position corresponding to nucleotide 1003 of SEQ ID NO:1 or as an A-444C single nucleotide polymorphism in the leukotriene C4 synthase gene. In addition, the SNP in the leukotriene C4 synthase gene can be described as an A→C transversion at the position corresponding to nucleotide 13 of SEQ ID NO:2.

Accordingly, in one embodiment, the present invention provides a method of identifying a subject as responsive to treatment of an inflammatory disease with one or more PUFAs, comprising detecting, in a nucleic acid sample of the subject having an inflammatory disease, the presence of C allele at single nucleotide polymorphism (SNP) rs730012, wherein the presence of said C allele at rs730012 in said nucleic acid sample from the subject identifies the subject as being responsive to treatment of said inflammatory disease with one or more PUFAs.

As used herein, “responsive,” “responsiveness” (and grammatical variations thereof) to treatment with one or more PUFAs, means a desirable or favorable response to the treatment (e.g., an alleviation of symptoms of the disease is observed). Thus, in some aspects of the invention, responsiveness of a subject having, for example, asthma, to treatment with one or more PUFAs can mean an improvement in the subject's forced expiratory volume (FEV). In other embodiments, responsiveness of a subject having, for example, diabetes mellitus Type 2 and/or metabolic syndrome, to treatment with one or more PUFAs, can mean modulation of a subject's insulin production.

The term “modulate,” “modulates” or “modulation” refers to enhancement (e.g., an increase) or inhibition (e.g., a reduction) in the specified activity.

In contrast to the predictive value of identifying a C allele at rs 30012 (heterozygous (C/A) or homozygous (C/C)), it is noted that subjects who are homozygous for an A allele at rs730012 (A/A) do not respond to treatment with one or more PUFAs in a statistically significant manner and thus it is not predictable whether an inflammatory disease and/or inflammation of that subject will be responsive to treatment with one or more PUFAs. Thus, in some cases, a subject homozygous for the A allele at rs 730012 may be responsive to treatment with one or more PUFAs and in other cases, a subject homozygous for the A allele at rs 730012 may not be responsive to treatment with one or more PUFAs.

The PUFAs can be provided as a PUFA-based supplement, which means a supplement comprising one or more (e.g., 2, 3, 4, 5, 6 7, and the like) botanical-based ω-3 PUFAs, one or more marine based ω-3 PUFAs, one or more (e.g., 2, 3, 4, 5, 6, 7 and the like) botanical-based ω-6 PUFAs, and/or any combination thereof. The PUFA-based supplements of the invention can be provided in any form including, but not limited to, a dietary supplement, a medical food and/or a prescription product and can be administered in any manner that delivers to a subject an effective amount of the PUFAs (e.g., food, drink, parenteral feeding, injection, enteral feeding, dermal).

Marine based ω-3 PUFA are complex mixtures which includes, but are not limited to, eicosapentaenoic acid (EPA), docosapentaenoic acid (DPA), and/or docosahexaenoic acid (DHA). A marine-based ω-3 PUFA can be provided, for example, as a purified PUFA or as a complex oil derived from any source that produces the PUFA. Sources of marine-based ω-3 PUFAs include, but are not limited to, fish, krill, crab, shrimp, lobster, mussel, octopus, oyster, clam, and/or marine algae.

Similarly, botanical oils are complex mixtures of ω-3 PUFAs and/or ω-6 PUFAs. Non-limiting examples of botanical-based ω-3 PUFAs include stearidonic acid (SDA), alpha-linolenic acid (ALA), eicosapentaenoic acid (EPA), docosahexaenoic acid (DHA), and/or docosapentaenoic acid (DPA). Botanical-based ω-6 PUFAs include, but are not limited to, gamma-linolenic acid (GLA) and/or dihomogamma-linolenic acid (DGLA). A botanical-based PUFA can be provided, for example, as a purified PUFA or as an oil derived from any source that produces the PUFA. Sources of botanical-based ω-3 PUFAs and ω-6 PUFAs include, but are not limited to, borage, echium, evening primrose, flaxseed, canola, walnut (e.g., black, English, Persian), soybean, oat, hickory nut, butternut, beechnut, chia seed, marine algae and/or any combination thereof. Additional sources of PUFAs include microbial oils (e.g., bacterial, fungal), which can be used alone or in combination with PUFAs derived from the same source or any other source.

Thus, the one or more PUFAs can be, but are not limited to, stearidonic acid (SDA), alpha-linolenic acid (ALA), gamma-linolenic acid, eicosapentaenoic acid (EPA), docosapentaenoic acid (DPA), docosahexaenoic acid (DHA), gamma-linolenic acid (GLA), dihomogamma-linolenic acid (DGLA), or any combination thereof, which can be provided to a subject in any form including as a PUFA-based supplement comprising, consisting essentially of consisting of, one or more purified PUFAs, as a combination of the sources known to produce the one or more PUFAs (e.g., fish, echium, borage, flaxseed, marine algae, and the like), and/or as an oil derived from one or more sources known to produce said PUFAs.

The genetic marker of the invention (an adenine to cytosine transversion 444 nucleotides upstream from the first codon in LTC4S (A-444C)) is correlated with responsiveness of an inflammatory disease of a subject to treatment with polyunsaturated fatty acids (PUFAs) as described herein and as disclosed in the Examples provided herein for correlating genetic markers with various phenotypic traits. In general, identifying such a correlation involves conducting analyses that establish a statistically significant association and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

As described herein, in some embodiments, the methods of the invention can include making a correlation between genetic markers in the leukotriene C4 synthase gene and responsiveness of a subject's inflammatory disease to treatment with a PUFA-based supplement. Thus, patients who respond well to treatment with a PUFA-based supplement can be analyzed for specific genetic markers in the leukotriene C4 synthase gene (e.g., an A>C transversion at -444 in the leukotriene C4 synthase gene; a C allele at rs730012) and a correlation can be established according to the methods provided herein. A subject who is a candidate for treatment for an inflammatory disease can be assessed for the presence of the appropriate genetic markers (e.g., C/A, C/C) and it can be determined if treatment with a PUFA-based supplement is appropriate (i.e., if the patient will be responsive to treatment with a PUFA-based supplement).

A “subject” of the invention includes any animal that is susceptible to an inflammatory disease or to inflammation as described herein. Nonlimiting examples of subjects of this invention include mammals, such as humans, nonhuman primates, domesticated mammals (e.g., dogs, cats, rabbits, guinea pigs, rats), livestock and agricultural mammals (e.g., horses, cows, pigs, goats). In other embodiments, a subject may additionally be an animal such as a bird or reptile. Thus, in some embodiments, a subject can be any domestic, commercially or clinically valuable animal. Subjects may be male or female and may be any age including neonate, infant, juvenile, adolescent, adult, and/or geriatric subjects. Thus, in some embodiments, a subject of this invention is a mammalian subject. In particular embodiments, a subject is a human subject. A human subject of this invention can be of any age, gender, race or ethnic group (e.g., Caucasian (white), Asian, African, black, African American, African European, Hispanic, Mideastern, etc.).

A “subject in need thereof” is a subject known to have, or suspected of having, diagnosed with, or at risk of having an inflammatory disease or inflammation. A subject of this invention can also include a subject not previously known or suspected to have inflammatory disease or inflammation and/or in need of treatment for inflammation and/or an inflammatory disease. For example, a subject of this invention can be administered the compositions of this invention even if it is not known or suspected that the subject has an inflammation and/or an inflammatory disease (e.g., prophylactically). A subject of this invention is also a subject known or believed to be at risk of developing inflammation and/or an inflammatory disease as described herein.

As used herein, an “inflammatory disease” includes, but is not limited to, asthma, allergy including, but not limited to, atopic dermatitis, urticaria, allergic rhinitis and/or allergic rhinoconjunctivitis, psoriasis, acute myocardial infarction, glomerulonephritis, inflammatory bowel disease, irritable bowel syndrome, chronic joint disease including, but not limited to, rheumatoid arthritis and/or osteoarthritis, diabetes mellitus Type 2, metabolic syndrome, obesity, heart disease including, but not limited to, atherosclerosis and/or cardiovascular disease, stroke, cancer (e.g., breast cancer, colon cancer, prostate cancer), an autoimmune disease including, but not limited to, systemic lupus erythematosus and/or Crohn's disease, sepsis, toxic shock, organ failure, organ transplant, IgA nephropathy, brain disorders including, but not limited to, schizophrenia, and/or depression, diseases associated with loss of cognitive function including, but not limited to, Alzheimer's disease and age related cognitive decline, cystic fibrosis, menstrual discomfort, cyclic breast pain, premature labor, gout, venous leg ulcers, coronic urticaria, primary dysmenorrhea, endometriosis, and/or Lyme disease. In some embodiments, the inflammatory disease is asthma, allergy, cardiovascular disease, chronic joint disease, diabetes, metabolic syndrome, and/or any combination thereof. In some particular embodiments, the inflammatory disease is asthma. In still other embodiments, the inflammatory disease is diabetes.

In the methods described herein, the detection of a genetic marker in a subject can be carried out according to methods well known in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York). For example, DNA is obtained from any suitable sample from the subject that will contain DNA and the DNA is then prepared and analyzed according to well-established protocols for the presence of genetic markers according to the methods of this invention. In some embodiments, analysis of the DNA can be carried out by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, or by electrophoresis. Thus, the present invention further provides oligonucleotides for use as primers and/or probes for detecting and/or identifying genetic markers according to the methods of this invention.

In other aspects, the present invention provides a method of identifying, within a population of subjects having an inflammatory disease, a subpopulation of subjects having an inflammatory disease that is responsive to treatment with one or more PUFAs (e.g., a PUFA-based supplement), comprising detecting (e.g., by amplification), in a nucleic acid sample from each of said subjects, the presence of a C allele at single nucleotide polymorphism (SNP) rs730012 (A-444C), wherein the subpopulation of subjects having an inflammatory disease that is responsive to treatment with one or more PUFAs comprises those subjects having a C allele at rs730012, thereby identifying, within a population of subjects having an inflammatory disease, a subpopulation of subjects having an inflammatory disease that is responsive to treatment with one or more PUFAs.

Additionally provided is a method of predicting the response of a subject having an inflammatory disease to treatment with one or more PUFAs, comprising detecting the presence of a C allele at single nucleotide polymorphism (SNP) rs730012 in a nucleic acid sample from said subject, wherein the presence of a C allele at rs730012 the subject as being responsive to treatment with one or more PUFAs.

In still other aspects, the present invention provides a method of determining a suitable treatment for an inflammatory disease in a subject in need thereof, comprising detecting in a nucleic acid sample of the subject in need thereof the presence of a C allele at single nucleotide polymorphism (SNP) rs730012, wherein the C allele at rs730012 is correlated with responsiveness to treatment with one or more PUFAs, thereby identifying a subject for which treatment with one or more PUFAs is a suitable treatment.

In additional aspects, the present invention provides a method of treating an inflammatory disease in a subject in need thereof with one or more PUFAs (e.g., a PUFA-based supplement), comprising (a) detecting, in a nucleic acid sample from the subject, the presence of a C allele at single nucleotide polymorphism (SNP) rs730012, wherein the presence of said C allele at rs730012 in said nucleic acid sample identifies the subject as being responsive to treatment of said inflammatory disease with one or more PUFAs; and (b) administering an effective amount of one or more PUFAs to the subject identified to be responsive to treatment with one or more PUFAs, thereby treating an inflammatory disease in a subject in need thereof.

In other aspects of the present invention a method of treating inflammation in a subject in need thereof with one or more PUFAs (e.g., a PUFA-based supplement) is provided, comprising (a) detecting, in a nucleic acid sample from the subject, the presence of a C allele at single nucleotide polymorphism (SNP) rs730012, wherein the presence of said C allele at rs730012 in said nucleic acid sample identifies the subject as being responsive to treatment of said inflammation with one or more PUFAs; and (b) administering an effective amount of one or more PUFAs to the subject identified to be responsive to treatment with one or more PUFAs, thereby treating the inflammation in a subject in need thereof.

In further aspects of the invention, treating an inflammatory disease and/or inflammation in a subject in need thereof comprises administering to said subject an effective amount of one or more PUFAs. As discussed above, the one or more PUFAs can be administered in any form or manner effective to achieve a therapeutic and/or beneficial effect.

Thus, as a further aspect, the invention provides pharmaceutical formulations and methods of administering the same to achieve a therapeutic and/or beneficial effect. In representative embodiments, the pharmaceutical formulation may comprise stearidonic acid (SDA), alpha-linolenic acid (ALA), eicosapentaenoic acid (EPA), docosapentaenoic acid (DPA), docosahexaenoic acid (DHA), gamma-linolenic acid (GLA), and/or dihomogamma linolenic acid (DGLA), alone or in any combination, in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like

The PUFA(s) of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the PUFA(s) (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

Thus, in some aspects of the invention, SDA can be administered in an amount from about 25 mg to about 10 g, ALA can be administered in an amount from about 25 mg to about 10 g, EPA can be administered in an amount from about 25 mg to about 10 g, DPA can be administered in an amount from about 25 mg to about 10 g, DHA can be administered in an amount from about 25 mg to about 10 g, GLA can be administered in an amount from about 25 mg to about 10 g and/or DGLA can be administered in an amount from about 25 mg to about 10 g. Thus, in some embodiments, the SDA, ALA, EPA, DPA, DHA, GLA, and/or DGLA can each be administered in an amount of about 10 mg, 15 mg, 20 mg, 25 mg, 30 mg, 35 mg, 40 mg, 45 mg, 50 mg, 55 mg, 60 mg, 65, mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1 g, 1.1 g, 1.2 g, 1.3 g, 1.4 g, 1.5 g, 1.6 g, 1.7 g, 1.8 g, 1.9 g, 2.0 g, 2.1 g, 2.2 g, g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.6 g, 6.7 g, 6.8 g, 6.9 g, 7.0 g, 7.1 g, 7.2 g, 7.3 g, 7.4 g, 7.5 g, 7.6 g, 7.7 g, 7.8 g, 7.9 g, 8.0 g, 8.1 g, 8.2 g, 8.3 g, 8.4 g, 8.5 g, 8.6 g, 8.7 g, 8.8 g, 8.9 g, 9.0 g, 9.1 g, 9.2 g, 9.3 g, 9.4 g, 9.5 g, 9.6 g, 9.7 g, 9.8 g, 9.9 g, 10.0 g, and/or any range therein, and/or any combination thereof.

In some embodiments, the SDA, ALA, EPA, DPA, DHA, GLA, and/or DGLA can each be administered to a subject in an amount of about 400 mg to about 2 g. In some particular embodiments, SDA (in echium) and GLA (in borage) are each provided to a subject in an amount in a range of about 400 mg to about 2 g. In some additional embodiments, GLA, EPA and SDA are administered to a subject in an amount as described herein. In further embodiments, GLA, EPA and SDA are each administered to a subject in an amount of about 400 mg to about 2 g. In still further embodiments, ALA, SDA and GLA are each administered to a subject in an amount as described herein. In particular embodiments, ALA, SDA and GLA are each administered to a subject in an amount of about 400 mg to about 2 g.

Thus, as described herein, one or more PUFAs (e.g., SDA, ALA, DPA, DHA, GLA, DGLA, and/or EPA) can be used to treat an inflammatory disease in a subject identified as responsive to such treatment. In some embodiments of the invention, the one or more PUFAs can be comprised in a single composition and/or formulation and administered as such to the subject. In other embodiments, the one or more PUFAs can be comprised in any combination in more than one (e.g., 2, 3, 4, 5, 6, 7, and the like) composition and/or formulation, and therefore can be administered to a subject as more than one composition/formulation.

The polyunsaturated fatty acids of the invention (e.g., SDA, ALA, DPA, DHA, GLA, DGLA and/or EPA) can be administered as free fatty acids. In other embodiments, the PUFAs are administered as fatty acyl esters. Fatty acyl esters of the invention include, but are not limited to, diglycerides, triglycerides, ethyl esters, phospholipids, steryl esters, and sphingolipids.

The PUFAs can be administered to a subject in any suitable form including but not limited to a dietary supplement, a medical food and/or a prescription product. As used herein, a “medical food” means a food that is specifically formulated and intended for the dietary management of a disease that has distinctive nutritional needs that cannot be met by normal diet alone. Such foods can be ingested orally or administered through a feeding tube (i.e., enteral administration). Thus, in some embodiments of this invention, the PUFAs can be provided in a medical food. In further embodiments of the invention, the PUFAs can be administered as a medical food through a feeding tube.

For oral administration, the PUFA(s) can be administered in solid dosage forms, such as capsules, tablets, and powders, or in liquid dosage forms, such as elixirs, syrups, and suspensions. The PUFAs can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The formulations and compositions of the invention can also be provided as a dietary supplement. The dietary supplement can comprise the formulations or compositions of the present invention in the form of gel capsules or tablets and/or it can comprise the formulations or compositions of the present invention in the form of ingestible liquids. Such ingestible liquids can include, for example, fruit juices and milk based liquids that can be fortified with the compositions of the invention (e.g., PUFAs of the invention, for example, SDA, ALA, DPA, DHA, GLA, DGLA and/or EPA). In other embodiments, the formulations of the present invention can be incorporated into non-liquid foodstuffs.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution of the compound (i.e., PUFAs).

The PUFAs can alternatively be formulated for nasal administration or otherwise administered to the lungs of a subject by any suitable means, but is preferably administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159. Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the PUFAs can likewise be produced with any solid particulate medicament aerosol generator, by techniques known in the pharmaceutical art.

Alternatively, one can administer the PUFAs in a local rather than systemic manner, for example, in a depot or sustained-release formulation.

Further, the present invention provides liposomal formulations of the PUFAs disclosed herein and salts thereof. The technology for forming liposomal suspensions is well known in the art. When the PUFA or salt thereof is an aqueous-soluble salt, using conventional liposome technology, the same can be incorporated into lipid vesicles. In such an instance, due to the water solubility of the PUFA or salt, the PUFA or salt will be substantially entrained within the hydrophilic center or core of the liposomes. The lipid layer employed can be of any conventional composition and can either contain cholesterol or can be cholesterol-free. When the PUFA or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the PUFAs disclosed herein or salts thereof, can be lyophilized to produce a lyophilizate which can be reconstituted with a pharmaceutically acceptable carrier, such as water, to regenerate a liposomal suspension.

In particular embodiments of the invention, more than one administration (e.g., two, three, four, or more administrations) can be employed over a variety of time intervals (e.g., hourly, daily, weekly, monthly, etc.) to achieve therapeutic effects.

Treatment can be short-term (e.g., acute; for hours or days) or can be a long-term, chronic regimen (e.g., weeks, months or years). In some instances, the treatment is a maintenance regimen that lasts for months, years or even the life of the subject.

In further aspects, the present invention provides a kit for carrying out the methods of this invention, wherein the kit can comprise primers, probes, primer/probe sets, reagents, buffers, etc., as would be known in the art, for the detection of a mutation within LTC4S in a nucleic acid sample from the subject. Such a kit can further comprise blocking probes, labeling reagents, blocking agents, restriction enzymes, antibodies (e.g., secondary antibodies), ligands, immunoglobulin binding agents, sampling devices, positive and negative controls, etc., as would be well known to those of ordinary skill in the art.

In further aspects, the present invention provides a kit for carrying out the methods of this invention, wherein the kit can comprise oligonucleotides (e.g., primers, probes, primer/probe sets, etc.), reagents, buffers, etc., as would be known in the art, for the detection of the polymorphisms and/or alleles of the invention in a nucleic acid sample. For example, a primer or probe can comprise a contiguous nucleotide sequence that is complementary (e.g., fully (100%) complementary or partially (50%, 60%, 70%, 80%, 90%, 95%, etc.) complementary) to a region comprising the rs730012 SNP of the leukotriene CT4 synthase gene. In particular embodiments, a kit of this invention will comprise primers and probes that allow for the specific detection of the alleles of the invention. Such a kit can further comprise blocking probes, labeling reagents, blocking agents, restriction enzymes, antibodies, sampling devices, positive and negative controls, etc., as would be well known to those of ordinary skill in the art. Thus, in some embodiments, the present invention provides a kit comprising oligonucleotides to detect the C allele of single nucleotide polymorphism rs730012 in a nucleic acid sample.

### Additional Definitions

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of (and grammatical variants) means that the scope of a claim is to be interpreted to encompass the specified materials or steps recited in the claim and those that do not materially alter the basic and novel characteristic(s)” of the claimed invention. Thus, the term “consisting essentially of” when used in a claim of this invention is not intended to be interpreted to be equivalent to “comprising.”

The term “genetic marker” or “polymorphism” refers to a characteristic of a nucleotide sequence (e.g., in a chromosome) that is identifiable due to its variability among different subjects (i.e., the genetic marker or polymorphism can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein, etc., as would be well known to one of ordinary skill in the art). Polymorphisms may or may not have functional consequences.

A “single nucleotide polymorphism” (SNP) in a nucleotide sequence is a genetic marker that is polymorphic for two (or in some case three or four) alleles. A SNP can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation), and if a different polypeptide sequence is produced, the alleles of that SNP are non-synonymous. SNPs that are not in protein coding regions can affect expression and protein production through, for example, gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

An “allele” as used herein refers to one of two or more alternative forms of a nucleotide sequence at a given position (locus) on a chromosome. Alleles can be nucleotides present in a nucleotide sequence that makes up the coding or the non-coding region of a gene. An individual's genotype for a given gene is the set of alleles it happens to possess. In some embodiments of the invention, an individual identified as responsive to treatment with a combination of SDA and GLA, a combination of EPA and GLA, a combination of GLA, EPA and SDA, a combination of ALA, SDA and GLA, and/or any other combination of PUFAs described herein or as known in the art, is homozygous for the C allele at rs730012 (i.e., C/C). In other embodiments, an individual identified as responsive to treatment with a combination of SDA and GLA, a combination of EPA and GLA, a combination of GLA, EPA and SDA, a combination of ALA, SDA and GLA, and/or any other combination of PUFAs described herein or as known in the art, is heterozygous at rs730012 (i.e., C/A).

“Effective amount” as used herein refers to an amount of a composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder, prevention or delay of the onset of the disorder, and/or change in clinical parameters, disease or illness, etc., as would be well known in the art. In a representative embodiment, in a subject having asthma, the term “treat,”, “treating” or “treatment of” (and grammatical variations thereof) refer to an increase in forced expiratory volume (FEV) and/or a delay in the reduction of FEV, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to avoidance, prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent and/or delay the disease, disorder and/or clinical symptom in the subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The present invention is more particularly described in the following examples that are intended as illustrative only since numerous modifications and variations therein will be apparent to those skilled in the art.

### EXAMPLES

### Example 1

### Detection of the rs730012 Allele (A/C) in Subjects Having Asthma and Association of Response to Treatment with Botanical Oils

A randomized crossover study of Echium/borage supplementation was carried out in moderate stable asthmatic subjects.
Primary endpoint; change in forced expiratory volume in one second FEV₁
Secondary endpoints; symptom scores, medication use, changes in urinary leukotrienes

Genotyping was performed at the LTC4S locus for A to C variant. DNA was isolated from citrated blood using routine molecular biology methods as described herein. Genotyping of the LTC4S SNP (rs730012) was performed using the TaqMan probe-based, 5′ nuclease allelic discrimination assay.

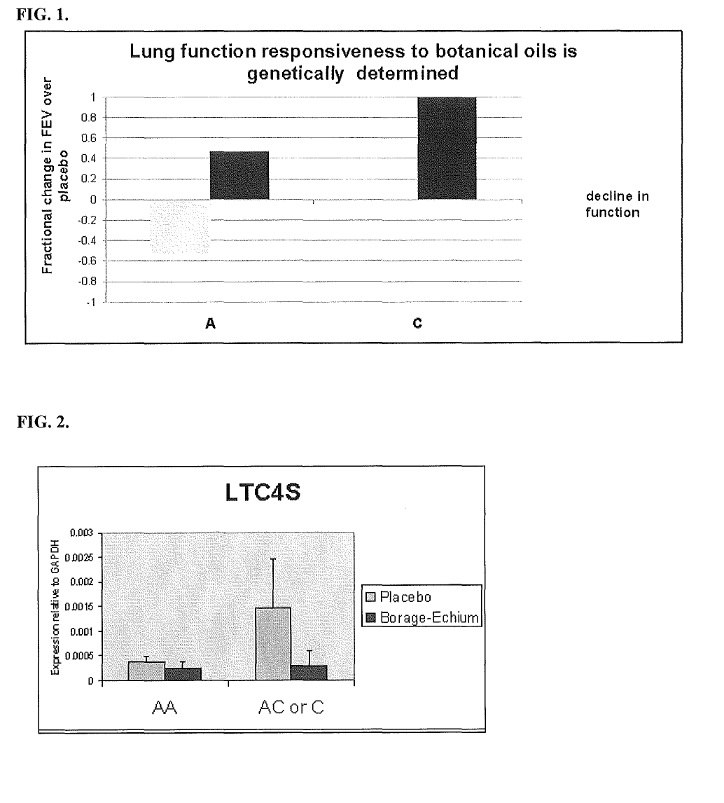

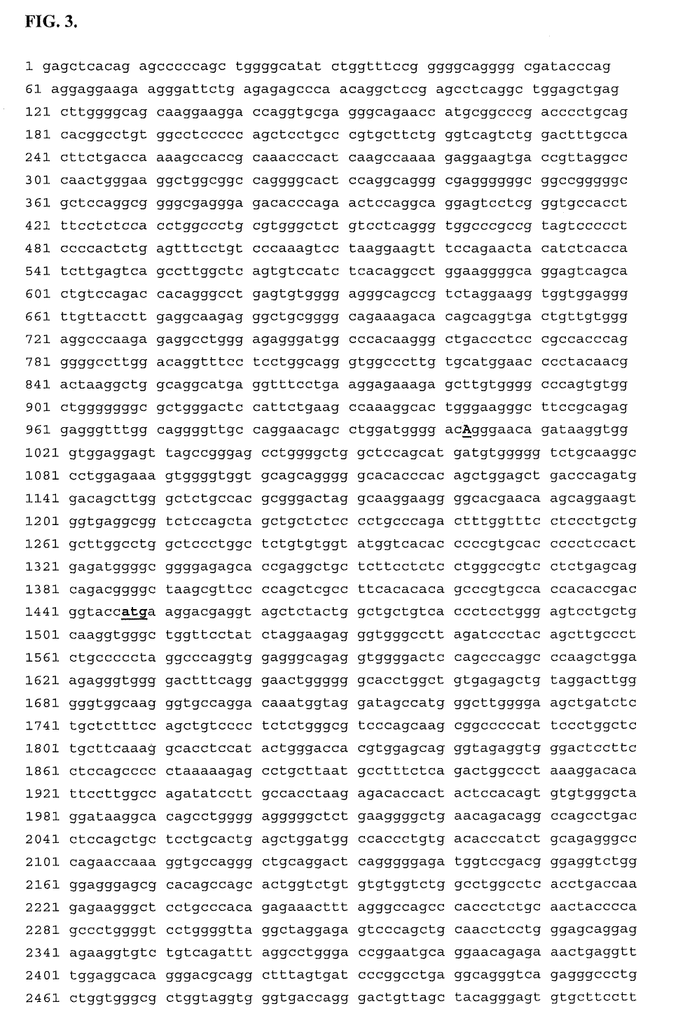

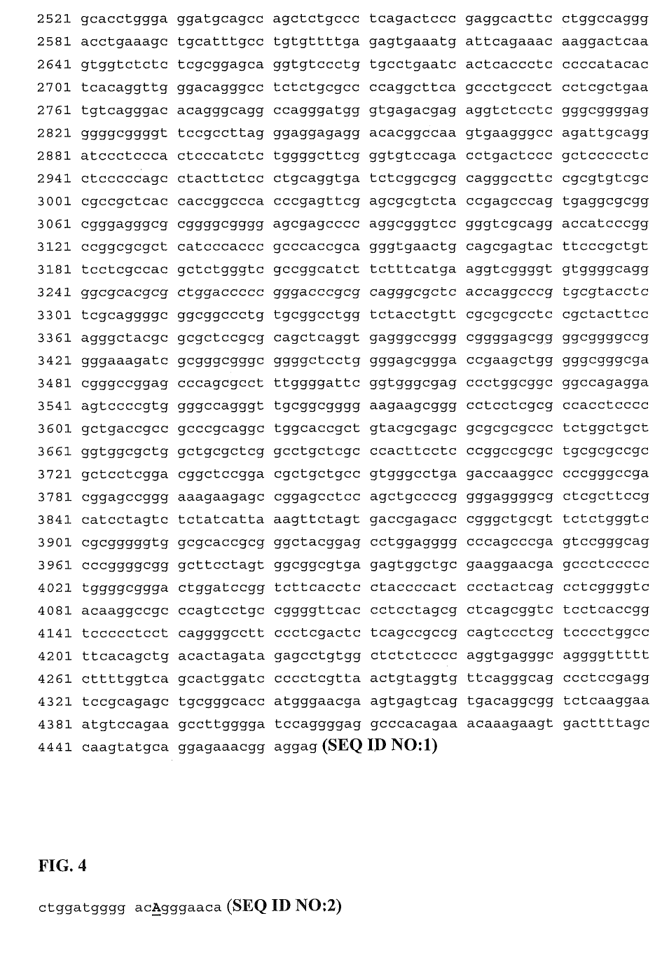

FIG. 1 shows the proportion of individuals whose pulmonary function improved or worsen on the borage/echium supplementation. These data show that 50% of asthmatic subjects with the AA (homozygous) genotype improved and 50% with the AA genotype worsened with the echium/borage supplementation, and thus overall there was no statistical improvement of the botanical oil combination in the AA genotype group. In contrast, all subjects with a C allele at rs730012 showed improvement in their pulmonary function. This was highly significant with a p<0.0007 and pointed out a strong genotypic effect at the LTC4S locus.

### Example 2

### Expression Levels of the Leukotriene CT4 Synthase Gene (mRNA) in Asthmatic Individuals

The level of expression of the leukotriene CT4 synthase gene (mRNA) was compared in asthmatic individuals with the A allele of rs730012 versus to asthmatic individuals having the C allele of rs730012. LTC4S is the enzyme that makes leukotrienes such as LTC4 that are responsible for much of the drop in pulmonary function seen in asthmatic patients. Messenger RNA was measured in individuals with A and C alleles to determine the impact of these in the SNP rs730012 on the expression of the LTC4S gene.

Messenger RNA levels of the LTC4 transcript were measured in circulating peripheral blood mononuclear cells. These cells were isolated from heparinized whole blood by density centrifugation and extracted with TRIzol (Invitrogen). RNA was isolated by routine molecular biology methods (as described herein), cleaned of contaminating genomic DNA, reverse transcribed to cDNA and subjected to real-time PCR (using commercially available primers and reagents) to evaluate the message RNA level of LTC4S relative to a reference gene (GAPDH).

The results are provided in FIG. 2, which shows that asthmatic individuals, homozygous or heterozygous for the C allele of rs730012 have much higher levels of the mRNA that codes for LTC4S than asthmatic individuals that are homozygous for the A allele at rs730012 indicating that those individuals homozygous or heterozygous for the C allele likely have more LTC4S protein to make more leukotrienes. FIG. 2 further shows that borage/echium oil supplementation reduced message levels of LTC4S in asthmatic subjects with the C allele to levels seen in the AA subjects. Since AA subjects did not naturally contain high message levels, there was little effect of the borage/echium combination in these subjects. Without being bound to any particular theory, this may be provide an explanation for the observation that borage/echium oil supplementation has much a more efficacious effect on pulmonary function of asthmatic subjects homozygous or heterozygous for the C allele than those with the AA genotype.

### Example 3

### Detection of the rs730012 Allele (A/C) in Subjects Having Diabetes and Association of Response to Treatment with Botanical Oils

To evaluate the efficacy of a combination of borage seed oil and echium seed oil in reducing the inflammatory components of metabolic syndrome and diabetes (Type 2) and to compare the efficacy of these plant-based oils to that of fish oils, a single blind, placebo-controlled trial is carried out. Participants are randomized into one of three groups: Group 1 receives supplements containing a combination of borage seed oil and echium seed oil that has been shown to be effective in reducing inflammation. Group 2 receives supplements containing fish oils and serves as a positive control for down regulation of inflammation by EPA. Group 3 receives supplements containing corn oils and serves as the placebo group.

Genotyping was performed at LTC4S locus for the A to C variant. DNA was isolated from citrated blood using routine molecular biology methods as described herein. Genotyping of the LTC4S SNP (rs730012) was performed using the TaqMan probe-based, 5′ nuclease allelic discrimination assay. Blood collected at baseline at 4 weeks into the intervention and at the end of the intervention (8 weeks) is analyzed for changes in serum fatty acids and several biomarkers of glucose regulation and inflammation including fasting glucose, fasting insulin, leptin, Hb1Ac, hsCRP, adiponectin, TNFa, IL-6, IL-8, IL-10, IL-12, IL-17, as well as a CBC diff and a lipid panel.

1. A method of identifying a subject as responsive to treatment of an inflammatory disease with one or more polyunsaturated fatty acids (PUFAs), comprising:
detecting, in a nucleic acid sample of a subject having an inflammatory disease, the presence of a C allele at single nucleotide polymorphism rs730012,
wherein the presence of said C allele at rs730012 in said nucleic acid sample from the subject identifies the subject as being responsive to treatment of said inflammatory disease with one or more PUFAs.

2. A method of identifying, within a population of subjects having an inflammatory disease, a subpopulation of subjects having an inflammatory disease that is responsive to treatment with one or more polyunsaturated fatty acids (PUFAs), comprising:
detecting, in a nucleic acid sample from each of said subjects, the presence a C allele at single nucleotide polymorphism rs730012,
wherein the subpopulation of subjects having an inflammatory disease that is responsive to treatment with one or more PUFAs comprises those subjects having a C allele at rs730012, thereby identifying, within a population of subjects having an inflammatory disease, a subpopulation of subjects having an inflammatory disease that is responsive to treatment one or more PUFAs.

3. A method of identifying a subject as responsive to treatment of inflammation with one or more polyunsaturated fatty acids (PUFAs), comprising:
detecting, in a nucleic acid sample of a subject having inflammation, the presence of a C allele at single nucleotide polymorphism rs730012,
wherein the presence of said C allele at rs730012 in said nucleic acid sample from the subject identifies the subject as being responsive to treatment of said inflammation with one or more PUFAs.

4. A method of determining a suitable treatment for an inflammatory disease in a subject in need thereof, comprising
detecting, in a nucleic acid sample of the subject, the presence of a C allele at single nucleotide polymorphism rs730012,
wherein the C allele of rs730012 is correlated with responsiveness to one or more polyunsaturated fatty acids (PUFAs), thereby identifying a subject for which treatment with one or more PUFAs is a suitable treatment.

5. The method of claim 4, further comprising administering to a subject in which the C allele at rs730012 is detected, an effective amount of one or more PUFAs.

6. A method of treating an inflammatory disease and/or inflammation in a subject in need thereof with one or more polyunsaturated fatty acids (PUFAs), comprising:
(a) detecting, in a nucleic acid sample from the subject, the presence of a C allele at single nucleotide polymorphism rs730012, wherein the presence of said C allele at rs730012 in said nucleic acid sample identifies the subject as being responsive to treatment of said inflammatory disease and/or inflammation with one or more PUFAs; and
(b) administering an effective amount of one or more PUFAs to the subject identified to be responsive to treatment with one or more PUFAs, thereby treating an inflammatory disease and/or inflammation in a subject in need thereof.

7. The method of claim 6, wherein the one or more PUFAs is selected from the group of PUFAs consisting of a botanical-based ω-3 PUFA, a marine based ω-3 PUFA, a botanical-based ω-6 PUFA, or any combination thereof.

8. The method of claim 7, wherein the botanical-based PUFA is provided in the form of an oil derived from a source selected from the group consisting of borage, echium, flaxseed, canola, walnut (black, English, Persian), soybean, oat, hickory nut, butternut, beechnut, chia seed, marine algae, and any combination thereof, and/or the marine-based PUFA is provided in the form of an oil derived from a source selected from the group consisting of fish, krill, crab, shrimp, lobster, mussel, octopus, oyster, clam, and any combination thereof.

9. The method of claim 7, wherein a botanical-based ω-3 PUFA is stearidonic acid (SDA), alpha-linolenic acid (ALA), or any combination thereof, a marine based ω-3 PUFA is eicosapentaenoic acid (EPA), docosapentaenoic acid (DPA), docosahexaenoic acid (DHA), or any combination thereof, and a botanical-based ω-6 PUFA is gamma-linolenic acid (GLA), dihomogamma-linolenic acid (DGLA), or any combination thereof.

10. The method of claim 9, wherein SDA is administered in an amount from about 25 mg to about 10 g, ALA is administered in an amount from about 25 mg to about 10 g, EPA is administered in an amount from about 25 mg to about 10 g, DPA is administered in an amount from about 25 mg to about 10 g, DHA is administered in an amount from about 25 mg to about 10 g, GLA is administered in an amount from about 25 mg to about 10 g, and/or DGLA is administered in an amount from about 25 mg to about 10 g.

11. The method of claim 9, wherein SDA is administered in an amount from about 400 mg to about 2 g, ALA is administered in an amount from about 400 mg to about 2 g, EPA is administered in an amount from about 400 mg to about 2 g, DPA is administered in an amount from about 400 mg to about 2 g, DHA is administered in an amount from about 400 mg to about 2 g, GLA is administered in an amount from about 400 mg to about 2 g, and/or DGLA is administered in an amount from about 400 mg to about 2 g.

12. The method of claim 10, wherein the SDA, ALA, EPA, DPA, DHA, GLA and/or DGLA are administered as free fatty acids.

13. The method of claim 10, wherein the SDA, ALA, EPA, DPA, DHA GLA and/or DGLA are administered as fatty acyl esters.

14. The method of claim 13, wherein said esters are selected from the group consisting of diglycerides, triglycerides, ethyl esters, phospholipids, steryl esters, and sphingolipids.

15. The method of claim 6, wherein the one or more PUFAs are provided as a combination of SDA and GLA, a combination of GLA, EPA and SDA, or a combination of ALA, SDA and GLA.

16. The method of claim 6, wherein said detecting comprises performing a hybridization assay.

17. The method of claim 6, wherein said detecting comprises performing a nucleic acid amplification assay.

18. The method of claim 6, wherein the inflammatory disease is asthma, allergy, cardiovascular disease, chronic joint disease, diabetes mellitus Type 2 and/or metabolic syndrome.

19. The method of claim 6, wherein the inflammatory disease is asthma.

20. The method of claim 6, wherein the inflammatory disease is diabetes mellitus Type 2.

21. The method of claim 6, wherein the subject is mammalian.

22. The method of claim 21, wherein the mammalian subject is human.

23. A kit for identifying a subject as responsive to treatment of an inflammatory disease and/or inflammation with one or more polyunsaturated fatty acids (PUFAs), comprising oligonucleotides to detect the C allele of single nucleotide polymorphism rs730012 in a nucleic acid sample from the subject.


SEQUENCE LISTING (2 sequences)

SEQ ID NO: 1 - DNA, 4465 bp, Homo sapiens
gagctcacagagcccccagctggggcatatctggtttccgggggcaggggcgatacccagaggaggaagaagggattctgagagagcccaacaggctccgagcctcaggctggagctgagcttggggcagcaaggaaggaccaggtgcgagggcagaaccatgcggcccgacccctgcagcacggcctgtggcctcccccagctcctgcccgtgcttctgggtcagtctggactttgccacttctgaccaaaagccaccgcaaacccactcaagccaaaagaggaagtgaccgttaggcccaactgggaaggctggcggccaggggcactccaggcagggcgaggggggcggccgggggcgctccaggcggggcgagggagacacccagaactccaggcaggagtcctcgggtgccacctttcctctccacctggccctgcgtgggctctgtcctcagggtggcccgccgtagtccccctccccactctgagtttcctgtcccaaagtcctaaggaagtttccagaactacatctcaccatcttgagtcagccttggctcagtgtccatctcacaggcctggaaggggcaggagtcagcactgtccagaccacagggcctgagtgtggggagggcagccgtctaggaaggtggtggagggttgttaccttgaggcaagagggctgcggggcagaaagacacagcaggtgactgttgtgggaggcccaagagaggcctgggagagggatggcccacaagggctgaccctcccgccacccagggggccttggacaggtttcctcctggcagggtggcccttgtgcatggaacccctacaacgactaaggctggcaggcatgaggtttcctgaaggagaaagagcttgtggggcccagtgtggctgggggggcgctgggactccattctgaagccaaaggcactgggaagggcttccgcagaggagggtttggcaggggttgccaggaacagcctggatggggacagggaacagataaggtgggtggaggagttagccgggagcctggggctggctccagcatgatgtgggggtctgcaaggccctggagaaagtggggtggtgcagcagggggcacacccacagctggagctgacccagatggacagcttgggctctgccacgcgggactaggcaaggaaggggcacgaacaagcaggaagtggtgaggcggtctccagctagctgctctcccctgcccagactttggtttcctccctgctggcttggcctggctccctggctctgtgtggtatggtcacacccccgtgcaccccctccactgagatggggcggggagagcaccgaggctgctcttcctctcctgggccgtcctctgagcagcagacggggctaagcgttccccagctcgccttcacacacagcccgtgccaccacaccgacggtaccatgaaggacgaggtagctctactggctgctgtcaccctcctgggagtcctgctgcaaggtgggctggttcctatctaggaagagggtgggccttagatccctacagcttgccctctgccccctaggcccaggtggagggcagaggtggggactccagcccaggcccaagctggaagagggtggggactttcagggaactggggggcacctggctgtgagagctgtaggacttgggggtggcaagggtgccaggacaaatggtaggatagccatgggcttggggaagctgatctctgctctttccagctgtcccctctctgggcgtcccagcaagcggcccccattccctggctctgcttcaaaggcacctccatactgggaccacgtggagcagggtagaggtgggactccttcctccagccccctaaaaagagcctgcttaatgcctttctcagactggccctaaaggacacattccttggccagatatccttgccacctaagagacaccactactccacagtgtgtgggctaggataaggcacagcctggggagggggctctgaaggggctgaacagacaggccagcctgacctccagctgctcctgcactgagctggatggccaccctgtgacacccatctgcagagggcccagaaccaaaggtgccagggctgcaggactcagggggagatggtccgacgggaggtctggggagggagcgcacagccagcactggtctgtgtgtggtctggcctggcctcacctgaccaagagaagggctcctgcccacagagaaactttagggccagcccaccctctgcaactaccccagccctggggtcctggggttaggctaggagagtcccagctgcaacctcctgggagcaggagagaaggtgtctgtcagatttaggcctgggaccggaatgcaggaacagagaaactgaggtttggaggcacagggacgcaggctttagtgatcccggcctgaggcagggtcagagggccctgctggtgggcgctggtaggtgggtgaccagggactgttagctacagggagtgtgcttccttgcacctgggaggatgcagccagctctgccctcagactcccgaggcacttcctggccagggacctgaaagctgcatttgcctgtgttttgagagtgaaatgattcagaaacaaggactcaagtggtctctctcgcggagcaggtgtccctgtgcctgaatcactcaccctcccccatacactcacaggttgggacagggcctctctgcgccccaggcttcagccctgccctcctcgctgaatgtcagggacacagggcaggccagggatgggtgagacgagaggtctcctcgggcggggagggggcggggttccgccttagggaggagaggacacggccaagtgaagggccagattgcaggatccctcccactcccatctctggggcttcgggtgtccagacctgactcccgctccccctcctcccccagcctacttctccctgcaggtgatctcggcgcgcagggccttccgcgtgtcgccgccgctcaccaccggcccacccgagttcgagcgcgtctaccgagcccagtgaggcgcggcgggagggcgcggggcggggagcgagccccaggcgggtccgggtcgcaggaccatcccggccggcgcgctcatcccacccgcccaccgcagggtgaactgcagcgagtacttcccgctgttcctcgccacgctctgggtcgccggcatcttctttcatgaaggtcggggtgtggggcaggggcgcacgcgctggacccccgggacccgcgcagggcgctcaccaggcccgtgcgtacctctcgcaggggcggcggccctgtgcggcctggtctacctgttcgcgcgcctccgctacttccagggctacgcgcgctccgcgcagctcaggtgagggccgggcggggagcggggcggggccggggaaagatcgcgggcgggcggggctcctggggagcgggaccgaagctgggggcgggcgacgggccggagcccagcgcctttggggattcggtgggcgagccctggcggcggccagaggaagtccccgtggggccagggttgcggcggggaagaagcgggcctcctcgcgccacctccccgctgaccgccgcccgcaggctggcaccgctgtacgcgagcgcgcgcgccctctggctgctggtggcgctggctgcgctcggcctgctcgcccacttcctcccggccgcgctgcgcgccgcgctcctcggacggctccggacgctgctgccgtgggcctgagaccaaggcccccgggccgacggagccgggaaagaagagccggagcctccagctgccccggggaggggcgctcgcttccgcatcctagtctctatcattaaagttctagtgaccgagacccgggctgcgttctctgggtccgcgggggtggcgcaccgcgggctacggagcctggaggggcccagcccgagtccgggcagcccggggcgggcttcctagtggcggcgtgagagtggctgcgaaggaacgagccctccccctggggcgggactggatccggtcttcacctcctaccccactccctactcagcctcggggtcacaaggccgcccagtcctgccggggttcaccctcctagcgctcagcggtctcctcaccggtccccctcctcaggggccttccctcgactctcagccgccgcagtccctcgtcccctggccttcacagctgacactagatagagcctgtggctctctccccaggtgagggcaggggtttttcttttggtcagcactggatccccctcgttaactgtaggtgttcagggcagccctccgaggtccgcagagctgcgggcaccatgggaacgaagtgagtcagtgacaggcggtctcaaggaaatgtccagaagccttggggatccaggggaggcccacagaaacaaagaagtgacttttagccaagtatgcaggagaaacggaggag

SEQ ID NO: 2 - DNA, 20 bp, Homo sapiens
ctggatggggacagggaaca